(19) United States
(12) Patent Application Publication
(10) Pub. No.: US 2026/0096780 A1
(43) Pub. Date: Apr. 9, 2026

(54) BAYESIAN DENOISING FOR RETROSPECTIVE DETECTION

(71) Applicant: Dexcom, Inc., San Diego, CA (US)

(72) Inventors: Andrea Facchinetti, Padova (IT); Simone Del Favero, Padova (IT); Giovanni Sparacino, Padova (IT); Elena Idi, Padova (IT); Eleonora Manzoni, Padova (IT); Nunzio Camerlingo, Padova (IT)

(21) Appl. No.: 18/908,001

(22) Filed: Oct. 7, 2024

(51) Int. Cl. A61B 5/00; A61B 5/145; G16H 10/40

(52) U.S. Cl. CPC A61B 5/7203; A61B 5/14532; A61B 5/7267; G16H 10/40

(57) ABSTRACT

In accordance with a method of detecting a pressure induced sensor artifact (PISA) in an analyte trace, a measured analyte trace having a plurality of data samples obtained over a period of time from an analyte sensor is received. A reconstructed analyte trace and an associated confidence window is generated from the measured analyte trace using a Bayesian denoising algorithm that includes a model that models the measured analyte trace as a sum of an unknown true analyte trace and a measurement error. The measured analyte trace is compared to the reconstructed analyte trace to identify data samples in the measured analyte trace that are located outside of the confidence window as being associated with a PISA.

### BACKGROUND

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also potential. Diet and exercise also affect blood glucose levels.

Diabetes conditions are sometimes referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become insulin resistant. The result is that even though insulin is present in the body, the insulin is not sufficiently used by the patient's body to effectively regulate blood sugar levels.

Devices, such as continuous glucose monitoring (CGM) sensors, can help to manage diabetes. However, the accuracy of such devices can be impacted by a variety of factors during use of the devices.

### SUMMARY

In one aspect, a method of detecting a pressure induced sensor artifact (PISA) in an analyte trace is presented. The method includes: receiving, by a processor, a measured analyte trace having a plurality of data samples obtained over a period of time from an analyte sensor; generating, by the processor, a reconstructed analyte trace and an associated confidence window from the measured analyte trace using a Bayesian denoising algorithm that includes a model that models the measured analyte trace as a sum of an unknown true analyte trace and a measurement error; and comparing, by the processor, the measured analyte trace to the reconstructed analyte trace to identify data samples in the measured analyte trace that are located outside of the confidence window as being associated with a PISA.

In an embodiment of the first aspect, the method further includes: repeating, by the processor, the generating of a reconstructed analyte trace and an associated confidence window a plurality of times using the measured analyte trace while excluding each time a different subset of the data samples to thereby generate a plurality of reconstructed analyte traces; and wherein the comparing includes comparing the measured analyte trace to each of the reconstructed analyte traces to identify at least a given one of the different subsets of the analyte samples as being associated with a PISA if a residual between the measured analyte trace and a reconstructed analyte trace generated by excluding the given one of the different subsets of the analyte samples exceeds a threshold.

In an embodiment of the first aspect, the Bayesian denoising algorithm includes a tunable regularization parameter that is tunable to produce a smoother reconstructed analyte trace as the regularization parameter varies by one of increasing or decreasing and to produce a reconstructed analyte trace that more closely follows the data samples in the measured analyte trace as the regularization parameter varies by the other of increasing or decreasing.

In an embodiment of the first aspect, the method further includes adjusting the tunable regularization parameter to an optimal value that increases detection of true positive PISAs and decreases detection of false positive PISAs.

In an embodiment of the first aspect, the tunable regularization parameter is adjusted for a particular measured analyte trace.

In an embodiment of the first aspect, the Bayesian denoising algorithm includes a tunable regularization parameter that is tunable to produce a smoother reconstructed analyte trace as the regularization parameter varies by one of increasing or decreasing and to produce a reconstructed analyte trace that more closely follows the data samples in the measured analyte trace as the regularization parameter varies by the other of increasing or decreasing.

In an embodiment of the first aspect, the method of further includes retuning the regularization parameter to decrease the regularization parameter by a scaling factor that is greater than zero and less than one to thereby increase sensitivity and decrease detection of false positive PISAs.

In an embodiment of the first aspect, the scaling factor is determined based on a population dataset.

In an embodiment of the first aspect, the method further includes adjusting a confidence window parameter that determines a width of the confidence window to balance detection of true positive PISAs and avoidance of detection of false positive PISAs.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 is a diagram conceptually illustrating an example continuous analyte monitoring system including example continuous analyte sensor(s) with sensor electronics, in accordance with certain aspects of the present disclosure.

FIG. 2 illustrates an example of a pressure induced sensor artifact (PISA) in continuous glucose monitoring (CGM) data.

FIG. 3 is a schematic block diagram of one example of a Bayesian denoising process in accordance with certain aspects of the present disclosure.

FIG. 4 illustrates the role of the regularization parameter γ in the derivation of the denoised CGM signal.

FIG. 5 is a flowchart illustrating one example of a PISA detection method in accordance with certain aspects of the present disclosure.

FIG. 6 illustrates an example of a model for describing the dynamics of compression artifacts in which a pressure of unitary amplitude and duration D is applied on a sensor.

FIG. 7 illustrates examples of different profiles of fault-free CGM signals and CGM signals corrupted by PISAs over time for a specific subject.

FIG. 8 is a graph comparing the number of false positives (FP)/day detected using the core Bayesian denoising algorithm and the missing samples approach using 3, 5 and 7 missing data samples.

FIG. 9 is a graph comparing the number of FP/day detected using both the best missing samples approach (5 missing data samples) and the scaling factor approach.

FIGS. 10(a) and 10(b) show the number of FP/day and the sensitivity, respectively, for every subject using an optimized algorithm that uses both the missing sample and the scaling approaches.

FIG. 11 (upper) is a table presenting parameters yielding the highest performance on a simulated training set and corresponding performance metrics and FIG. 11 (lower) is a graph comparing the number of FP/day that are detected in real-world data using the core Bayesian denoising algorithm, the missing samples approach, and the scaling approach.

FIG. 12 (upper) is a table presenting parameters and corresponding performance metrics for real-world data and FIG. 12 (lower) is a graph showing the distribution of the FP/day that are detected using a set of optimal parameters.

### DETAILED DESCRIPTION

Management of diabetes can present complex challenges for patients, clinicians, and caregivers, as a confluence of many factors can impact a patient's glucose level and glucose trends. To assist patients with better managing this condition, portable or wearable medical devices (e.g., sensors and other types of monitoring and diagnostic devices) as well as a variety of diabetes intervention software applications (hereinafter “applications”) have been developed by various providers.

Because CGM sensors are able to measure glucose concentration in a user, they are important for diabetes management. Analysis of CGM data is beneficial for improving diabetes therapies and, in turn, for improving overall glycemic control for a user. For example, analysis of trends and patterns in CGM data after a meal can help improve insulin dosing by suggesting rescue carbohydrate intake, evaluating glucose variability, quantifying the effectiveness of a therapy, and improving visualization of relevant CGM patterns.

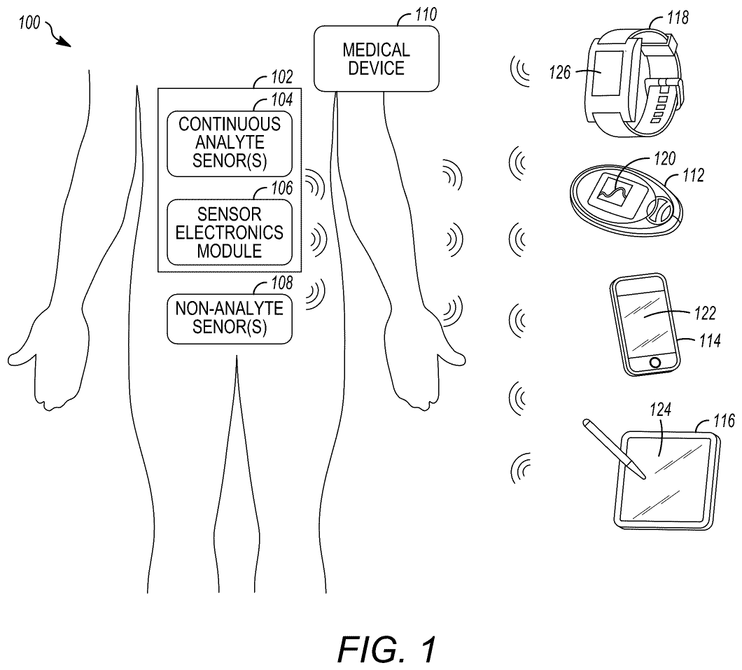

Unfortunately, CGM traces can be inaccurate. One source of uncertainty arises from random measurement error, which affects any signals obtained from a sensor. Another source of inaccuracy is that caused by large artifacts that can be occasionally generated during sensor functioning. FIG. 1 displays a portion of representative CGM data clearly affected by both these sources of error.

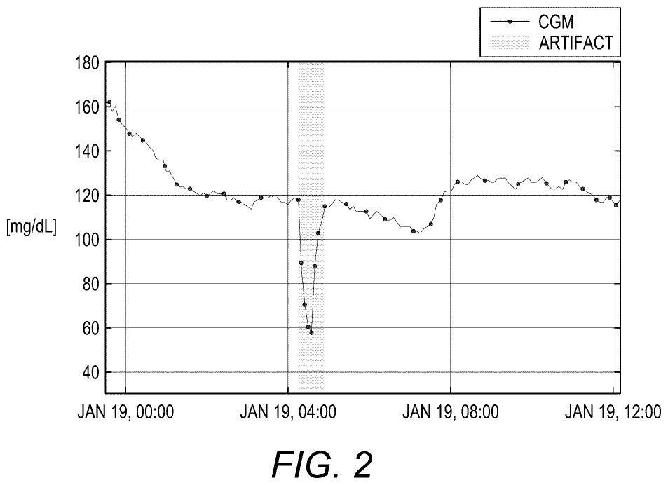

The presence of large artifacts in CGM traces can induce errors of clinical relevance. For instance, the spurious drop in BG highlighted in FIG. 2 by the shaded area, can be misinterpreted as a hypoglycemic event. Therefore, the preliminary retrospective detection and elimination of CGM data portions affected by errors and artifacts is important to minimize the risk of making incorrect clinical evaluations and therapy decisions.

The glucose measurement in CGM sensors is typically performed by a glucose-oxidase reaction on a small needle, usually inserted in the subcutaneous tissue of the abdomen or the upper back of the arm. Failures on the CGM sensor measurements are often related to the biomechanics of the sensor-tissue interface. Specifically, the mechanical compression of the sensor, for example triggered by the individual who unintentionally applies pressure on the sensor during sleep, alters the diffusion process at the needle insertion site. This alteration adversely affects the sensor's sensitivity, leading to a systematic underestimation of glucose concentration across multiple samples. The artifacts resulting from mechanical compression are commonly referred to as Pressure Induced Sensor Artifacts (PISAs). The drop in glucose levels that typically occurs at the beginning of PISA can be easily mistaken for a physiological fluctuation, making PISAs' detection a complex task, even for an expert human operator.

Studies have reported that pressure over the sensor insertion site led to a transient decrease in current. Notably, compression at the sensor insertion site has been implicated as a potential cause for anomalous hypoglycemic measurements, particularly during nighttime when users lie directly on the sensor. Moreover, many studies concerning animal trials illustrate acute pressure effects on CGM data.

Strategies aimed at addressing PISAs detection can be broadly categorized into two groups: data driven and model-based techniques. Model-based fault detection techniques use explicit models of patient physiology to predict the expected BG level under normal conditions. Data-driven approaches mainly exploit the availability of historical data and can be used for a variety of monitored processes. The performance of data-driven approaches heavily relies on training data, and possibly on labeled examples of faulty and fault-free data previously evaluated by an expert.

Unfortunately, the need for CGM, insulin injection, and meal data limits the applicability of these PISA detection approaches to a fraction of the TID population (typically those individuals using advanced diabetes technologies, such as sensor augmented pumps, automated insulin delivery systems or smart insulin pens).

Embodiments described herein detect PISAs with the exclusive use of CGM data, thereby avoiding the need for insulin and meal data. As described in more detail below, the CGM signal is modelled as the sum of the true noise free glucose profile (assumed to be unknown) and a colored measurement noise. A Bayesian denoising algorithm is used which leverages a-priori statistical models of the CGM signals to estimate the true glucose profile, along with its confidence interval or window. Each sample of the estimated profile is then compared with the CGM measurements: if a significant discrepancy is observed, even when accounting for the confidence interval of the estimate, the sample is identified as being affected by PISA. As further described below, the PISA detection method is assessed in terms of retrospective detection using both simulated data, generated by the FDA-accepted UVa/Padova Type 1 Diabetes simulator, and a real-world dataset collected using the commercial Dexcom G6 sensor. Results demonstrate the effectiveness of the PISA detection techniques described herein.

Although the PISA detection techniques presented herein are described for illustrative purposes in terms of the detection of PISAs in CGM data obtained from a CGM sensor, more generally the systems and techniques described herein are applicable to the detection of PISAs in other analyte data obtained from other types of analyte sensors, such as insulin, oxygen, lactate, ketone, pyruvate, and/or potassium sensors, and/or from multi-analyte sensors that obtain analyte data related to two or more analytes.

Accordingly, the embodiments described herein provide systems and methods of detecting PISAs in analyte data. In particular, the embodiments herein provide a health management system, including a display device and an analyte monitoring system, including an analyte sensor (e.g., CGM sensor) configured to generate analyte measurements (e.g., glucose measurements) for transmission to the display device. The display device includes a processor configured to execute a software application for receiving and processing the analyte data (e.g., CGM data) indicative of the analyte measurements generated by the analyte sensor. The software application may use a PISA detection algorithm, such as the methods described herein, to detect PISAs in received analyte data. The software application may alternatively be configured to send the received analyte data to a server that executes the PISA detection algorithm, such as the methods described herein.

FIG. 1 illustrates an analyte monitoring system 100 including an example continuous analyte sensor system 102, non-analyte sensor(s) 108, medical device 110, and a plurality of display devices 112, 114, 116, and 118, in accordance with certain aspects of the present disclosure. The components of the analyte monitoring system 100 are configured to operate continuously to monitor one or more analytes of a user, in accordance with certain aspects of the present disclosure.

Continuous analyte monitoring system 102, in the illustrated embodiment, includes sensor electronics module 106 and one or more continuous analyte sensor(s) 104 (individually referred to herein as continuous analyte sensor 104 and collectively referred to herein as continuous analyte sensors 104) associated with sensor electronics module 106. Sensor electronics module 106 may be in wireless communication (e.g., directly or indirectly) with one or more of display devices 112, 114, 116, and 118. In certain embodiments, sensor electronics module 106 may also be in wireless communication (e.g., directly or indirectly) with one or more medical devices, such as medical devices 110 (individually referred to herein as medical device 110 and collectively referred to herein as medical devices 110), and/or one or more other non-analyte sensors 108 (individually referred to herein as non-analyte sensor 108 and collectively referred to herein as non-analyte sensor 108).

In certain embodiments, a continuous analyte sensor 104 may comprise a sensor for detecting and/or measuring analyte(s). The continuous analyte sensor 104 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 104 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 104 provides a data stream indicative of the concentration of one or more analytes in the user. The data stream may include raw data signals, which may then be converted into a calibrated and/or filtered data stream used to provide estimated analyte value(s) to the user.

In certain embodiments, continuous analyte sensor 104 may be a multi-analyte sensor, configured to continuously measure multiple analytes in a user's body. For example, in certain embodiments, the continuous multi-analyte sensor 104 may be a single multi-analyte sensor configured to measure two or more of glucose, insulin, lactate, ketones, pyruvate, and potassium in the user's body.

In certain embodiments, the continuous analyte sensor 104 may be a continuous glucose monitor (CGM). Some examples of a continuous glucose monitor include a glucose monitoring sensor. In some embodiments, glucose monitoring sensor is an implantable sensor, such as described with reference to U.S. Pat. No. 6,001,067 and U.S. Patent Publication No. US-2011-0027127-A1. In some embodiments, the glucose monitoring sensor is a transcutaneous sensor, such as described with reference to U.S. Patent Publication No. US-2006-0020187-A1. In yet other embodiments, the glucose monitoring sensor is a dual electrode analyte sensor, such as described with reference to U.S. Patent Publication No. US-2009-0137887-A1. In still other embodiments, the glucose monitoring sensor is configured to be implanted in a host vessel or extracorporeally, such as the sensor described in U.S. Patent Publication No. US-2007-0027385-A1. These patents and publications are incorporated herein by reference in their entirety.

As used herein, the term “continuous” may mean fully continuous, semi-continuous, periodic, etc. Such continuous monitoring of analytes is advantageous in diagnosing and staging a disease given the continuous measurements provide continuously up to date measurements as well as information on the trend and rate of analyte change over a continuous period. Such information may be used to make more informed decisions in the assessment of glucose homeostasis and treatment of diabetes.

In certain embodiments, sensor electronics module 106 includes electronic circuitry associated with measuring and processing the continuous analyte data, including prospective algorithms associated with processing and calibration of the analyte data. Sensor electronics module 106 can be physically connected to continuous analyte sensor(s) 104 and can be integral with (non-releasably attached to) or releasably attachable to continuous analyte sensor(s) 104. Sensor electronics module 106 may include hardware, firmware, and/or software that enables measurement of levels of analyte(s) via a continuous analyte sensor(s) 104. For example, sensor electronics module 106 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 112, 114, 116, and/or 118 are configured for displaying displayable analyte data, including analyte data, which may be transmitted by sensor electronics module 106. Each of display devices 112, 114, 116, or 118 can include a display such as a touchscreen display 120, 122, 124, or 126 for displaying analyte data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating analyte data to the user of the display device and/or receiving user inputs.

As described above, an analyte monitoring system 102 such as a CGM system is configured to continuously measure one or more analytes (e.g., glucose in a CGM system) and transmit the resulting analyte measurements, in the form of analyte data, to a display device (e.g., display device 112, 114, 116, and/or 118), which is configured with a PISA detection algorithm to detect PISAs associated with the analyte data before the analyte data is displayed to the user and/or analyzed for generating decision support recommendations. In certain embodiments, instead of a display device, the PISA detection algorithm may be executed on a server in data communication with the display device and/or continuous analyte monitoring system. In such embodiments, the server uses the PISA detection algorithm and transmits the results to the display device. In some embodiments, algorithms described herein may be implemented wholly or in-part on the display device, a server, and/or another device in communication with the display device and/or server.

### Bayesian Denoising

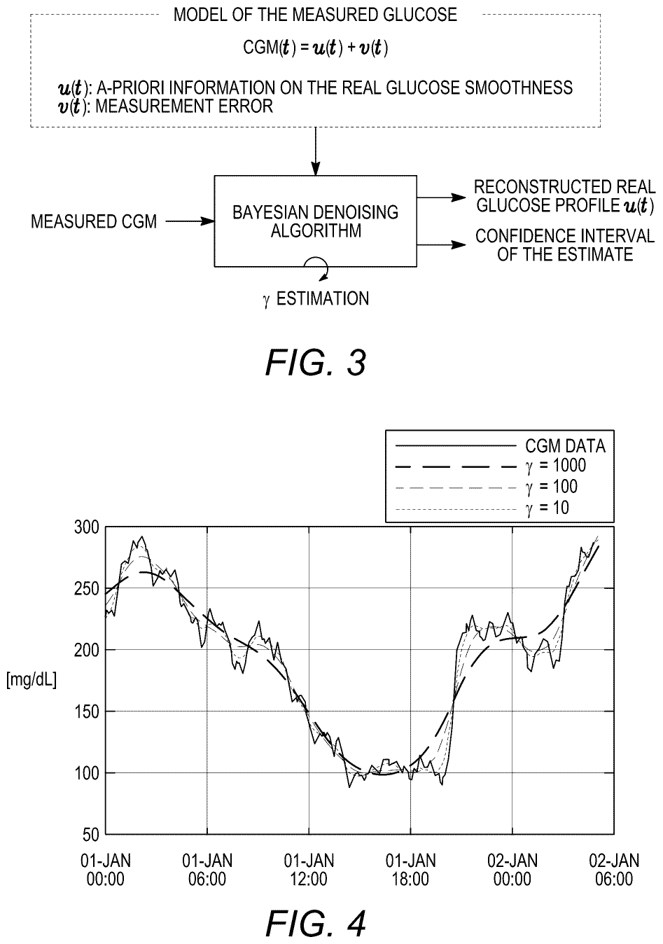

FIG. 3 shows a schematic block diagram of one example of the overall Bayesian denoising process, which for illustrative purposes will be described in more detail below for the particular case where the analyte is glucose. As shown, the Bayesian denoising algorithm receives the measured CGM trace (or other measured analyte trace) as input and uses the a-priori information on the unknown real glucose level and the measurement noise given by the model to provide at the output an estimate of the real glucose level and its confidence interval. The CGM signal may be the raw signal obtained from the CGM sensor or it may be a calibrated estimated glucose value (EGV). The algorithm can also recurrently estimate the regularization parameter γ.

To begin the modeling process, it is assumed that the artifact-free CGM signal can be modelled at each time t (which is a multiple of the sampling time T, which is illustrated herein as being 5 min), as the sum of two stochastic processes: one modeling the real and unknown glucose level u(t), and the other being an additive measurement error ν(t):

$$CGM ( t ) = u ( t ) + v ( t ) . \qquad ( 1 )$$

In order to estimate u(t) in a Bayesian embedding, an a priori statistical model of its variability is needed. As frequently done, the expected regularity of the true glucose level u(t) can be described using an integrated random walk model that consists of a double integration of a white noise process:

$$u ( t ) = 2 u ( t - 1 ) - u ( t - 2 ) + ω ( t ) , \qquad ( 2 )$$

where ω(t) represents the model noise, a zero-mean white noise with unknown variance λ², constant in time.

Generally, the noise corrupting CGM measurements presents a certain level of autocorrelation. Therefore, an autoregressive (AR) model of order 2 is used to describe the measurement noise ν(t):

$$v ( t ) = {a}_{1} v ( t - 1 ) + {a}_{2} v ( t - 2 ) + ε ( t ) , \qquad ( 3 )$$

where ε(t) is the zero-mean white noise guiding the AR process with unknown variance σ², constant in time. Moreover, in some embodiments, the correlation parameter may be set to a₁=1.3 and a₂=−0.42.

The reconstruction of the denoised u\hat{ }(t) profile is obtained retrospectively from the N CGM data samples collected by the sensor. The following N dimensional vectors are introduced:
CGM=[CGM(1), CGM(2), . . . , CGM(N)]^{T} containing the measured CGM samples.
U=[u(1), u(2), . . . , u(N)]^{T} containing the unknown real glucose levels to be estimated.
V=[ν(1), ν(2), . . . , ν(N)]^{T} containing the measurement noise sequence.

The linear minimum variance estimator of the unknown glucose levels U is:

$${U}^{\hat{}} = {( {A}^{T} A + γ {F}^{T} F )}^{- 1} {A}^{T} ACGM . \qquad ( 4 )$$

where γ, commonly referred to as the regularization parameter, is a real and non-negative parameter given by the ratio σ²/λ², while A^{T}A and F^{T}F are associated to (the inverse of) the variance of the measurement noise vector V and of the model noise vector U, respectively (see Appendix for details). As shown in FIG. 4, the regularization parameter γ determines the data fit vs signal smoothness trade-off. By raising the regularization parameter γ, the cost of smoothness increases and the data fitting becomes relatively less important. Conversely, by decreasing the value of the regularization parameter γ, the cost of smoothness decreases and the fidelity to the data becomes relatively more important.

Further comments on the criterion used in some embodiments to determine a numerical value for γₒₚₜ, can be found in the Appendix.

Moreover, it can be shown that the covariance matrix of the estimation error U^{˜}=U−U\hat{ } is given by:

$$Var ( {U}^{\sim} ) = {{σ}^{2} ( {A}^{T} A + γ {F}^{T} F )}^{- 1} . \qquad ( 5 )$$

Confidence intervals around the reconstructed glucose profile can be computed by leveraging Var (U^{˜}).

### Artifact Recognition

At each time t, the measured CGM signal CGM(t) is compared with the reconstructed glucose profile u\hat{ }(t), accounting also for the confidence intervals on the reconstruction. A PISA is suspected if the measurement exhibits a major deviation from the reconstructed profile. Specifically, since PISAs are expected to produce an abnormally low glucose reading, a CGM sample is labelled as affected by PISA if it falls below the lower limit of the confidence interval:

$$CGM ( t ) \leq {u}^{\hat{}} ( t ) - m std ( {u}^{\sim} ( t ) )$$

where std(u^{˜}(t)) represents the square root of the diagonal elements of the covariance matrix Var(U^{˜}) associated to the estimation error at time t. Moreover, m is a parameter that controls the width of the confidence interval or window.

An effective tuning of m can be important: choosing m too small will result to a narrow confidence interval, allowing prompt detection of PISAs, but also causing a high number of false alarms. Conversely, high values of m widen the confidence interval, avoiding false alarms, but at the same time limiting the detection of PISAs.

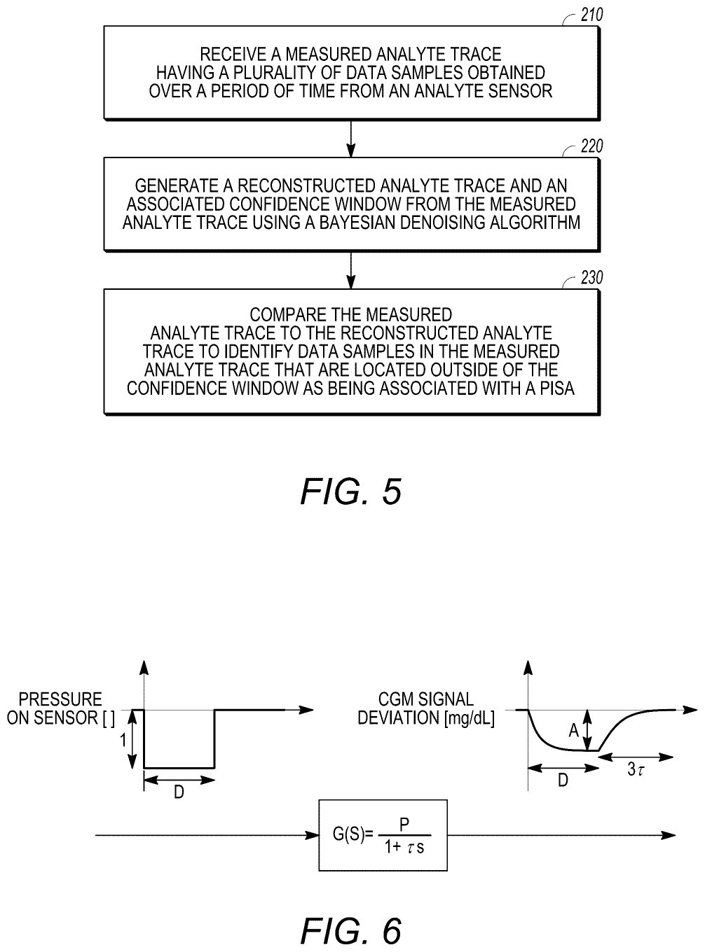

FIG. 5 is a flowchart illustrating one example of a PISA detection method in accordance with the subject matter herein. The method may be performed by a system (e.g., analyte monitoring system 100) that detects a pressure induced sensor artifact in an analyte signal. The system may include an analyte sensor system (e.g., analyte sensor system 102) configured to generate raw analyte data for a user. The system may include a memory comprising executable instructions and a processer (e.g., by a processor such as sensor electronics module 106, display devices 112, 114, 116, and/or 118, a server, etc.) in data communication with the memory and configured to execute the instructions to perform the various actions described herein.

Referring to FIG. 5, at block 210, a measured analyte trace having a plurality of data samples obtained over a period of time from an analyte sensor is received (e.g., by a processor such as sensor electronics module 106, display devices 112, 114, 116, and/or 118, a server, etc.). If the analyte is glucose, for example, in some implementations the data sample may be obtained from a continuous glucose monitor as described herein.

At block 220, a reconstructed analyte trace and an associated confidence window are generated (e.g., by a processor such as sensor electronics module 106, display devices 112, 114, 116, and/or 118, a server, etc.) from the measured analyte trace using a Bayesian denoising algorithm that includes a model that models the measured analyte trace as a sum of an unknown true analyte trace and a measurement error. The unknown glucose level may be estimated using an a priori statistical model of its variability and the measurement error may be estimated using an autoregressive function.

In some embodiments the Bayesian denoising algorithm used at block 220 includes a tunable regularization parameter that is tunable to produce a smoother reconstructed analyte trace as the regularization parameter varies (e.g., by increasing or decreasing) and to produce a reconstructed analyte trace that more closely follows the data samples in the measured analyte trace as the regularization parameter varies (e.g., the other one of increasing or decreasing). The tunable regularization parameter may be adjusted for a particular measured analyte trace. Additionally and/or alternatively, the tunable regularization parameter may be adjusted to an optimal value that increases detection of true positive PISAs and decreases detection of false positive PISAs. Additionally, the tunable regularization parameter may be retuned to decrease the regularization parameter by a scaling factor that is greater than zero and less than one to thereby increase sensitivity and decrease detection of false positive PISAs. In some cases the scaling factor may be determined based on a population dataset.

Returning to FIG. 5, after the reconstructed analyte trace is generated at block 220, the measured analyte trace is compared (e.g., by a processor such as sensor electronics module 106, display devices 112, 114, 116, and/or 118, a server, etc.) to the reconstructed analyte trace at block 230 to identify data samples in the measured analyte trace that are located outside of the confidence window as being associated with a PISA. The confidence window may be adjusted to balance detection of true positive PISAs and avoidance of detection of false positive PISAs. For example, the processor may adjust a confidence window parameter that determines a width of the confidence window to balance detection of true positive PISAs and avoidance of detection of false positive PISAs.

In some embodiments, the generation of the reconstructed analyte trace and the associated confidence window is repeated a plurality of times (e.g., two or more times) using the measured analyte trace, while excluding each time a different subset of the data samples to thereby generate two or more reconstructed analyte traces. The measured analyte trace is compared to each of the reconstructed analyte traces to identify at least one of the subsets of the analyte samples as being associated with a PISA if a residual between the measured analyte trace and a reconstructed analyte trace generated by excluding the subset of the analyte samples (e.g., different analyte samples) exceeds a threshold.

### Other Embodiments

To improve the fault detection performance, two additional embodiments of the Bayesian denoising algorithm are herein described, which are referred to as the missing sample (MS) approach, and the scaling (SC) approach. The Bayesian denoising algorithm described above, without use of the MS or SC approaches will be occasionally referred to herein as the core Bayesian denoising algorithm.

In the embodiment employing the missing sample approach, the core Bayesian denoising algorithm as described above is applied two or more times to the same CGM signal. At each iteration, a subset of #MS<<N consecutive CGM data samples (where #MS is a hyperparameter that can be tuned) is assumed to be missing, and thus not used in the denoising process. If the discarded data samples do not contain CGM data samples affected by a PISA, the resulting CGM signal estimate is expected to be similar to that obtained without discarding any data (i.e., with marginal discrepancy between the signal estimated with and without the missing sample approach). Conversely, if the discarded data samples contain CGM data samples affected by PISA, the resulting CGM signal estimate will be substantially different from that obtained without discarding any data samples (i.e., with a major discrepancy between the signal estimated with and without the missing sample approach). Indeed, discarding data containing faulty samples will lead to larger differences between the measured and estimated glucose values with respect to the application of the Bayesian denoising approach. As a consequence, faults will be easier to detect, and consequently the detection performance is expected to improve.

One straightforward and effective way that may be employed in some embodiments to discard a set of CGM samples in the reconstruction of the real glucose signal U is to set the corresponding diagonal elements of the covariance matrix of the measurement vector to a sufficiently high value. By doing so, the estimated real glucose signal U\hat{ } will be based almost exclusively on the a-priori information of the regularity of the true glucose level according to equation (2), and not on the actual value of the measured CGM data sample. It should be noted that the use of the Bayesian denoising algorithm with the missing sample approach has a higher computational cost that the use of the core Bayesian denoising algorithm alone because it performs a new smoothing iteration for each data sample in the CGM trace, each time excluding the samples considered as missing samples.

The other embodiment noted above, the so-called scaling approach, can be implemented to further increase the advantages of the missing sample approach, thus further improving detection performance.

In accordance with the scaling approach, the regularization parameter γ undergoes a-posteriori re-estimation of, and more particularly, a reduction of the optimal value of the regularization parameter γₒₚₜ previously estimated by the core Bayesian denoising algorithm. Specifically, by applying the denoising algorithm with the missing sample approach and using a scaled value of the optimal parameter γₒₚₜ, a reconstructed real glucose profile U\hat{ } can be realized which tends to follow the measured CGM data more closely. In particular,

$${γ}_{scaled} = {s}_{f} {γ}_{opt} , \qquad ( 6 )$$

where the scaling factor s_{f}∈(0, 1) needs to be tuned.

As a consequence, the discrepancy between the measured and estimated signals will be smaller in the data samples that are fault-free, thus reducing the false positive rate, while data portions corrupted by PISA will be affected only slightly. In this way, the reduction of false alarms can be accomplished, thus increasing the overall detection performance.

In summary, the core Bayesian denoising approach, the hyperparameter that requires estimation for each sensor-measured glucose trace is denoted as γₒₚₜ. When incorporating the missing sample approach, the number of missing samples (#MS) becomes an additional hyperparameter that needs tuning. Furthermore, in the scaling approach, the scaling factor s_{f} also requires tuning. Notably, while γₒₚₜ needs to be tuned for the specific CGM trace, both #MS and s_{f} are considered population parameters.

In some embodiments, an additional approach may be employed to reduce the detection of false positive PISAs. Because of the nature of the model employed in the Bayesian denoising approach, the model is sensitive to rapid changes in the slope of the CGM trace. This can cause systematic false positives in the case of rapid increases or decreases in the CGM trace, such as might occur after a meal. To reduce these false positives alerts, two optional steps may be implemented. First, if after detecting a suspected PISA in a portion of the CGM trace the CGM trace increases for more than some period of time, e.g., 40 minutes, the alert is ignored and treated as a false positive. Second, if after detecting a suspected PISA the CGM trace decreases for more than some period of time, e.g., 30 minutes, and the sum of the absolute values of the second derivative of the CGM trace (i.e., the cumulative sum of the rate of change of the slope of the CGM trace) over that period of time is lower than a threshold, the alert is ignored and treated as a false positive.

### Illustrative Validation of the Systems and Methods for Detecting PISAs

The following section illustrates that the systems and methods described herein are effective for retrospective detection of PISAs. To evaluate the PISA detection algorithm described herein it is important to test it on datasets with accurate labels for normal and anomalous conditions, which serve as ground truth. During real-life CGM use, it is unknown when the CGM trace is affected by PISA, so real-life datasets do not have these ground-truth labels.

Retrospective labelling can be performed by human-expert operators, but the process is time-consuming and unavoidably subject to errors. As an alternative, in-silico datasets from Type 1 Diabetes simulators can be used, allowing for precise fault simulation with known timing and duration. The limitation of this approach is that even the most advanced simulators are a simplified representation of the complex physiology of metabolic processes occurring in an individual and therefore the resulting dataset is less challenging than real-world data.

To obtain the best of both possible approaches, both in-silico datasets (UVa/Padova Type 1 Diabetes simulator) and a real-world dataset from TID adults using Dexcom G6 CGM sensors (Dexcom, Inc., San Diego, CA) were employed. While the simulated dataset is more numerous, the real-world dataset provides a diverse range of realistic faults.

### Simulated Dataset

The first dataset is obtained using the UVa/Padova Type 1 Diabetes Simulator, which is an accurate physiology simulator accepted by the US Food and Drug Administration as a substitute for animal testing prior to Artificial Pancreas clinical trials on humans. A recent version of the simulator was used, where new features have been added to increase the realism of the testing scenario, including intra-day variability of insulin sensitivity and time varying distributions of the subjects' therapy parameters.

For each of the 100 virtual adults considered in this study a total of 10 days of data were simulated (i.e. the average life expectancy of a CGM sensor), with 3 meals per day. Meal times were randomly chosen, with uniform distribution, in the time intervals of [7.00-8.00], [11.30-13.00], [18.30-20.00], respectively for breakfast, lunch and dinner. Meal amounts were randomly sampled, matching a previously developed distribution of the data, namely 58.2±22.5 g for breakfast, 77.7±27.0 g for lunch, 83.9±32.3 g for dinner. Since patients' estimation of the carbohydrate content in a meal may be inaccurate, the carbohydrate-counting error was simulated using a Gaussian distribution with zero mean and a variance derived using known techniques. Specifically, meals can be overestimated up to 150% and underestimated up to 75% of the actual carbohydrates content. A hybrid closed-loop insulin delivery therapy was simulated using a PID control algorithm.

For each of the 100 virtual subjects, a random number of PISA episodes is added to the 10 simulated days, following a truncated Gaussian distribution: the number of episodes is sampled from the Gaussian distribution with a mean equal to 3 and a standard deviation of 3 episodes in the 10 days, and resampled if found below the minimum of 1 episode or above the maximum of 10 episodes.

Each compression artifact is modelled as a temporary additive error a(t) that decreases the CGM signal. The additive artifact a(t) is obtained by filtering a rectangular signal with a duration D and a unitary amplitude, describing the presence of a mechanical pressure exerted on the sensor. The first-order linear system with a transfer function G(s)=P/(1+τs) models the delayed impact of the pressure on the glucose concentration. The maximum amplitude A reachable by the CGM signal can be easily demonstrated to be equal to A=P(1−e^{-D/τ}). Pressure duration D [min] and the system's time constant τ [min] are sampled from previously obtained distributions. The transfer function gain P [mgdL⁻¹] is sampled by a truncated version of the distribution to better match the amplitude observed in the analysis of the available real data. Specifically, P is truncated so that the minimum amplitude of the compression artifact is fixed to be 20 mgdL⁻¹ for episodes that last less than 30 min, while for longer faults it is fixed to 40 mgdL⁻¹, since longer compression artifacts typically have an overall larger amplitude.

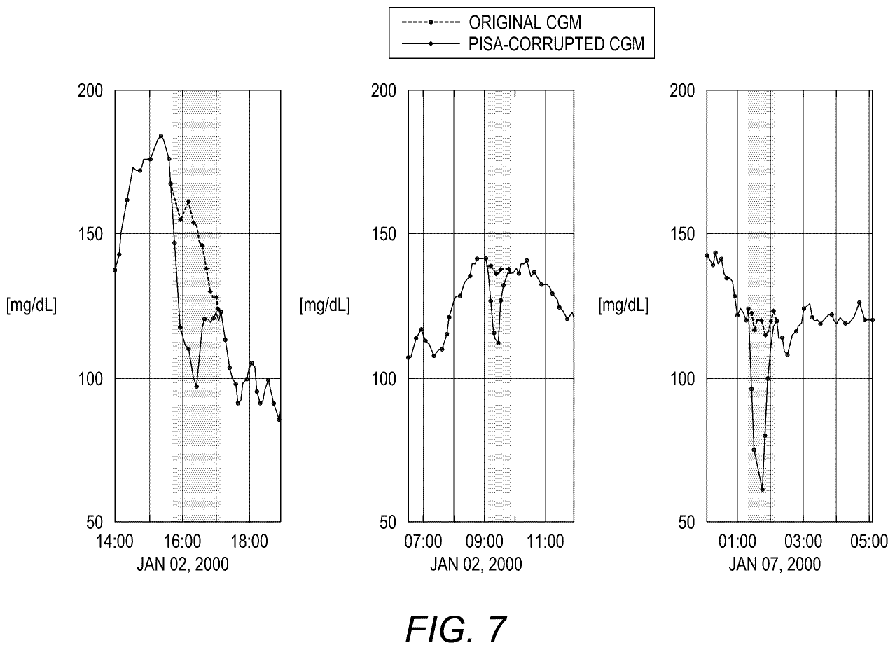

FIG. 6 shows the proposed model that is used to describe the dynamics of compression artifacts. FIG. 7 shows an example of different profiles of fault-free CGM signals and corrupted CGM signals that are corrupted by PISAs over time on a specific subject. The fault period is highlighted in the shaded area.

The dataset was then divided into training and test sets, with an 80-20 proportion on the subject level.

### Real-World Dataset

The real-world dataset includes 72 CGM traces collected over 36 adult subjects with TID who were wearing a pair of Dexcom G6 CGM sensors (Dexcom Inc., San Diego, CA), providing 1 sample every 5 min for a time interval of up to 10 days. Since each of the 36 subjects had 2 sensors on 2 different body locations, e.g. left arm and right arm, the comparison of the coupled CGM traces was used by human operators to label portions of the CGM traces as being either faulty or fault-free.

The labeling was performed in two steps. In the first step, 3 data analysts independently inspected the CGM traces to highlight possible PISAs. In the second step, an operator agreement meeting under the supervision of other 3 senior data analysts agreed on the final faulty or fault-free labels to be applied to portions of the CGM trace. The labeling process, even if performed with care, remains challenging, and affected by inter-operator variability.

Finally, it should be noted that simultaneous access to both the CGM traces collected in the same patient is permitted only during manual labelling. On the other hand, the retrospective PISA detection algorithms are tested using only one CGM trace.

### Assessment Criteria

The performance assessment of the PISA detection method described herein is as follows. If an alert is raised within the duration of the fault, a True Positive (TP) is assigned, otherwise, a False Negative (FN) is assigned. If an alert is generated but no fault actually occurred, a False Positive (FP) is assigned. For purposes of the particular type of anomaly detection being discussed herein, the consideration of True Negative (TN) events is of limited interest since it deals with a highly unbalanced dataset (e.g., only a few faults occurring over 10 days).

Then, the sensitivity (SE), also known as recall, is computed:

$$SE = 100 \frac{TP}{TP + FN} , \qquad ( 7 )$$

representing the percentage of faults correctly detected.

Moreover, the number of FPs per day of CGM use that each patient experienced is computed. The metric FP/day reports the average of this quantity over the population.

### Algorithm Tuning

The parameters of each method are tuned in the training set to minimize the cost function J of equation (8), which takes into account both SE and FP/day:

$$J = \sqrt{{( \frac{SE}{100} - 1 )}^{2} + {( FP / day )}^{2}} . \qquad ( 8 )$$

In fact the performance of each algorithm with fixed parameters can be represented as a point in (SE,FP/day) space. The optimal performance can be found on the bottom right corner, where SE=100% and FP/day=0. The cost function J quantifies the weighted deviation from this ideal performance, i.e., the Euclidean distance. It is important to consider that the sensitivity, as defined as a percentage in equation (7), needs to be scaled in order to be effectively compared with the FP/day metric.

### Results on Simulated Data

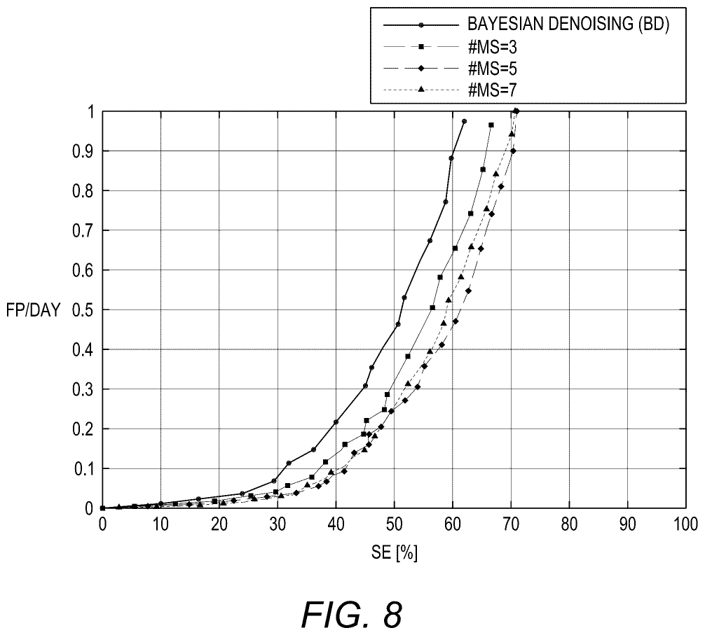

FIG. 8 presents a comparative analysis of the performance of the Bayesian denoising algorithm with and without use of the MS approach on the training set. Each point of the curve, which shows the dependence of the sensitivity (SE) on the FPs per day, corresponds to a different value of the parameter m. Moreover, the MS approach is applied using different missing samples: 3, 5, or 7. The curves in FIG. 6 show that the MS approach enhances the algorithm's ability to effectively detect faults. Notably, the optimal performance in terms of SE and FP/day is achieved with 5 missing samples, since this line approaches more closely the ideal performances (bottom right corner).

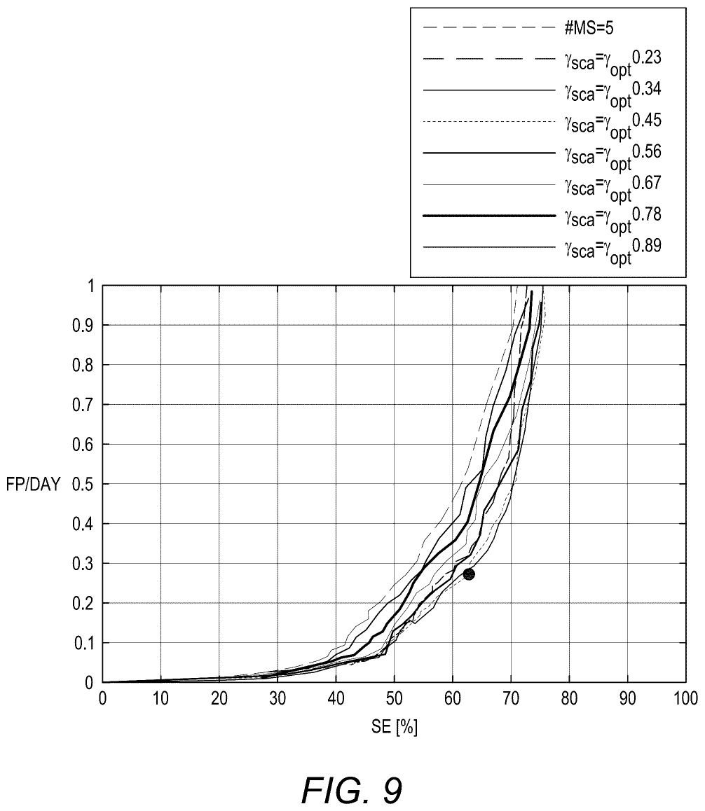

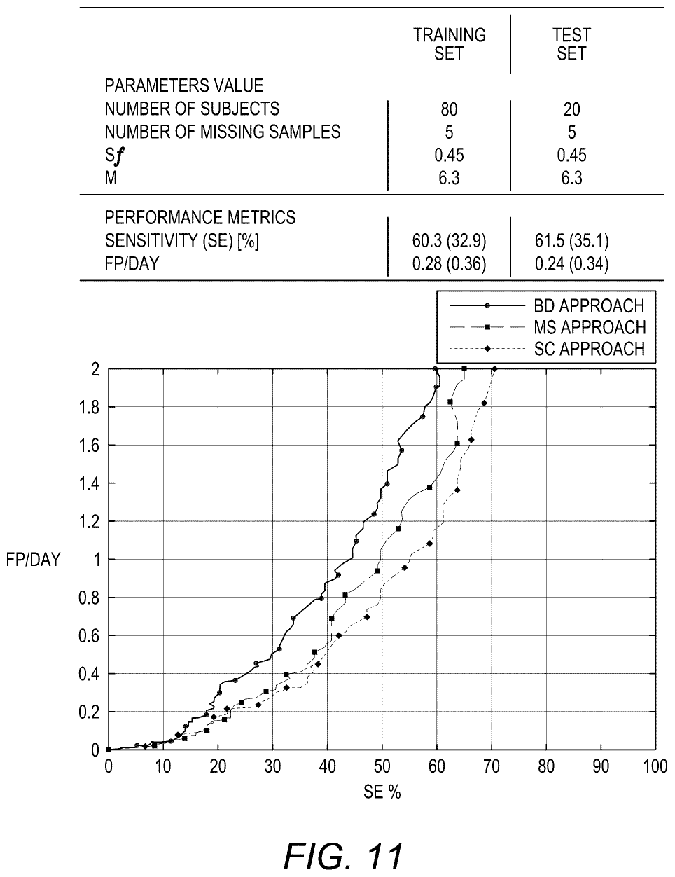

Next, the SC approach has been applied to the best MS technique (#MS=5) identified from FIG. 8. Various factors s_{f} are used to scale the previously estimated regularization parameter γₒₚₜ. The value of s_{f}=0.45 minimizes the Euclidean distance to the optimal point. The results are shown in FIG. 9, which like FIG. 8 shows the dependence of the sensitivity (SE) on the FPs per day. The black dot corresponds to the tuning minimizing the Euclidean distance. The parameters yielding the highest performance on the simulated training set are summarized in Table I, which is shown in the upper portion of FIG. 11.

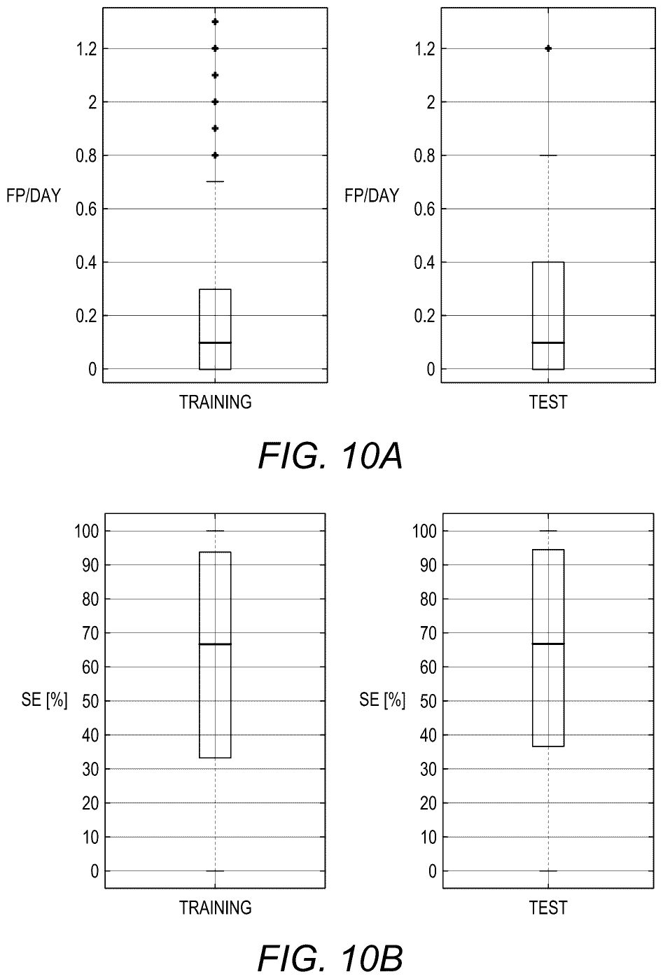

As a final step, the best performing set of hyperparameters obtained with the SC approach is tested on the test dataset. A performance of SE=61.5% and FP/day=0.24 is obtained. These results align with those observed in the training set. FIG. 10 shows the boxplots of the individual's performance metrics across the population for the optimal method, in both training and test sets. By looking at the FP/day metric, i.e., FIG. 10a, more than 75% of the patients exhibit less than 0.3 FP/day in the training set, while less than 0.4 in the test dataset. In both the training and test datasets, only a small fraction of outliers is found to have frequent false detection (>0.8 per day). Moreover, looking at the SE metric, i.e., FIG. 10b, only 25% of the patients exhibit a SE lower than 33% in the training dataset, while 25% of the patients exhibit a SE lower than 37% in the test dataset, showing that the training and test datasets are in good agreement.

### Results on Real-World Data

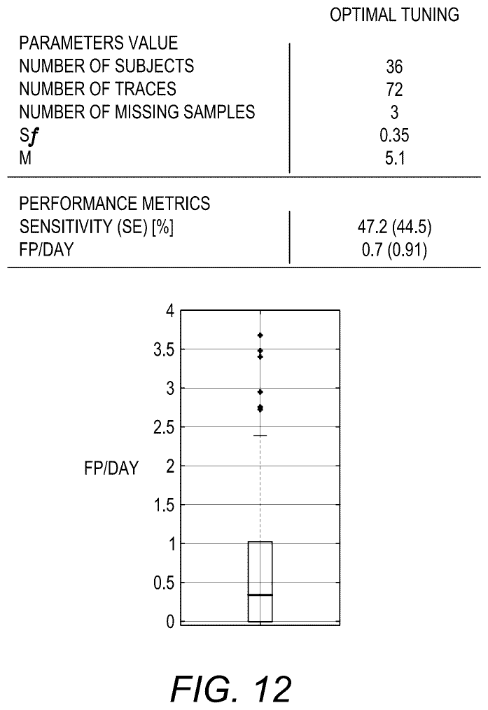

Real-world data are expected to be a more challenging and realistic benchmark since they are inherently more variable, affected by more confounding factors and other anomalies, such as inaccurate readings, missing samples, and shower artifacts. FIG. 11 (lower) shows the comparison of the three PISA detection embodiments, while the optimal performance is highlighted with the black square. As anticipated, the performance of the core Bayesian denoising algorithm has been enhanced through the use of the MS approach (in this dataset, the number of missing samples was reduced to #MS=3 to accommodate the artifact dynamics). By applying the scaling procedure and decreasing λ by 35%, the performance metrics further improve. The parameters and corresponding performance are summarized in Table II shown in the upper portion of FIG. 12.

FIG. 12 (lower) shows the distribution of the FP/day metric of the optimal method for each subject. More than 75% of the patients exhibit less than 1 FP/day and only a small fraction of outliers is found to have frequent false alarms (>2.4 per day).

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

The claimed subject matter may be implemented as a method, apparatus, or article of manufacture using standard programming and/or engineering techniques to produce software, firmware, hardware, or any combination thereof to control a computer to implement the disclosed subject matter. For instance, the claimed subject matter may be implemented as a computer-readable storage medium embedded with a computer executable program, which encompasses a computer program accessible from any computer-readable storage device or storage media. For example, computer readable storage media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). However, computer readable storage media do not include transitory forms of storage such as propagating signals, for example. Of course, those skilled in the art will recognize many modifications may be made to this configuration without departing from the scope or spirit of the claimed subject matter.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

### APPENDIX

### Covariance Matrices Derivation

The covariance of U=[u(1), u(2), . . . , u(N)]^{T}, the vector of N unknown glucose levels to be reconstructed and of V=[ν(1), ν(2), . . . , ν(N)]^{T}, the measurement noise sequence that affects it are now derived.
The unknown glucose levels are modelled as a double integrated random noise. Equation (2) can be rewritten in matrix form as

$$U = F ω$$

where: F is a square, lower triangular, Toeplitz matrix with first column containing the coefficients of the double integrated white noise, namely [1, −2, 1, 0, . . . , 0]^{T}; the vector ω=[ω(1), . . . , ω(N)] contains the zero-mean white noise of variance λ generating the process. As such, the variance of ω, Var[ω]=λI_{N×N}. Therefore

$$Var [ U ] = {F}^{- 1} Var [ ω ] {F}^{- T} = {λ ( {FF}^{T} )}^{- 1} .$$

The colored measurement noise ν corrupting the true glucose levels is modelled as an autoregressive (AR) model with structure reported in equation (3). It can be rewritten in matrix form as:

$$V = A ε$$

where: A is a square, lower triangular, Toeplitz matrix with first column containing information on the correlation parameters, equal to [1, a₁, a₂, 0, . . . , 0]^{T}; ε=[ε(1), . . . , ε(N)] contains the zero-mean white noise of variance σ generating the AR process. As such, the variance of ε, Var[σ]=σI_{N×N}. Therefore

$$Var [ V ] = {A}^{- 1} Var [ ε ] {A}^{- T} = {λ ( {AA}^{T} )}^{- 1}$$

### Optimization of the Regularization Parameter γ

Having defined the weighted residuals sum of squares (WRSS) in (11) as:

$$WRSS ( γ ) = {( CGM - {U}^{\hat{}} )}^{T} {A}^{T} A ( CGM - {U}^{\hat{}} ) , \qquad ( 9 )$$

and the weighed estimates sum of squares (WESS) as:

$$WESS ( γ ) = {U}^{\hat{} T} {F}^{T} {FU}^{\hat{}} , \qquad ( 10 )$$

it can be shown that

$$E [ WRSS ] = {σ}^{2} ( N - q ( {γ}_{opt} ) ) and E [ WESS ] = {λ}^{2} q ( {γ}_{opt} ) ,$$

where:

$$q ( γ ) = trace ( {{A}^{T} ( {A}^{T} A + γ {F}^{T} F )}^{- 1} A ) .$$

This suggests, for the current realization, to determine γ=σ²/λ², for the given CGM trace, by finding the value that satisfies:

$$\frac{WRSS ( γ )}{N - q ( γ )} = γ \frac{WESS ( γ )}{q ( γ )} , \qquad ( 11 )$$

with a fixed tolerance of 10⁻⁷.

What is claimed is:

1. A method of detecting a pressure induced sensor artifact (PISA) in an analyte trace, comprising:
receiving, by a processor, a measured analyte trace having a plurality of data samples obtained over a period of time from an analyte sensor;
generating, by the processor, a reconstructed analyte trace and an associated confidence window from the measured analyte trace using a Bayesian denoising algorithm that includes a model that models the measured analyte trace as a sum of an unknown true analyte trace and a measurement error; and
comparing, by the processor, the measured analyte trace to the reconstructed analyte trace to identify data samples in the measured analyte trace that are located outside of the confidence window as being associated with a PISA.

2. The method of claim 1, further comprising:
repeating, by the processor, the generating of a reconstructed analyte trace and an associated confidence window a plurality of times using the measured analyte trace while excluding each time a different subset of the data samples to thereby generate a plurality of reconstructed analyte traces; and
wherein the comparing includes comparing the measured analyte trace to each of the reconstructed analyte traces to identify at least a given one of the different subsets of the analyte samples as being associated with a PISA if a residual between the measured analyte trace and a reconstructed analyte trace generated by excluding the given one of the different subsets of the analyte samples exceeds a threshold.

3. The method of claim 1, wherein the Bayesian denoising algorithm includes a tunable regularization parameter that is tunable to produce a smoother reconstructed analyte trace as the regularization parameter varies by one of increasing or decreasing and to produce a reconstructed analyte trace that more closely follows the data samples in the measured analyte trace as the regularization parameter varies by the other of increasing or decreasing.

4. The method of claim 3, further comprising adjusting the tunable regularization parameter to an optimal value that increases detection of true positive PISAs and decreases detection of false positive PISAs.

5. The method of claim 3, wherein the tunable regularization parameter is adjusted for a particular measured analyte trace.

6. The method of claim 2, wherein the Bayesian denoising algorithm includes a tunable regularization parameter that is tunable to produce a smoother reconstructed analyte trace as the regularization parameter varies by one of increasing or decreasing and to produce a reconstructed analyte trace that more closely follows the data samples in the measured analyte trace as the regularization parameter varies by the other of increasing or decreasing.

7. The method of claim 6, further comprising retuning the regularization parameter to decrease the regularization parameter by a scaling factor that is greater than zero and less than one to thereby increase sensitivity and decrease detection of false positive PISAs.

8. The method of claim 7, wherein the scaling factor is determined based on a population dataset.

9. The method of claim 1, further comprising adjusting a confidence window parameter that determines a width of the confidence window to balance detection of true positive PISAs and avoidance of detection of false positive PISAs.

10. A system for detecting a pressure induced sensor artifact (PISA) in an analyte signal, the system comprising:
an analyte sensor system configured to generate raw analyte data for a user;
a memory comprising executable instructions; and
a processer in data communication with the memory and configured to execute the instructions to:
receive a measured analyte trace having a plurality of data samples obtained over a period of time from an analyte sensor;
generate a reconstructed analyte trace and an associated confidence window from the measured analyte trace using a Bayesian denoising algorithm that includes a model that models the measured analyte trace as a sum of an unknown true analyte trace and a measurement error; and
compare the measured analyte trace to the reconstructed analyte trace to identify data samples in the measured analyte trace that are located outside of the confidence window as being associated with a PISA.

11. (canceled)

12. A computer-readable medium comprising instructions which, when executed by a processor, cause the processor to perform a method for detecting a pressure induced sensor artifact (PISA) in an analyte signal, the method comprising:
receiving, by a processor, a measured analyte trace having a plurality of data samples obtained over a period of time from an analyte sensor;
generating, by the processor, a reconstructed analyte trace and an associated confidence window from the measured analyte trace using a Bayesian denoising algorithm that includes a model that models the measured analyte trace as a sum of an unknown true analyte trace and a measurement error; and
comparing, by the processor, the measured analyte trace to the reconstructed analyte trace to identify data samples in the measured analyte trace that are located outside of the confidence window as being associated with a PISA.

13. (canceled)

14. The system of claim 10, wherein the processor is further configured to execute the instructions to:
repeat the generating of a reconstructed analyte trace and an associated confidence window a plurality of times using the measured analyte trace while excluding each time a different subset of the data samples to thereby generate a plurality of reconstructed analyte traces; and
wherein the comparing includes comparing the measured analyte trace to each of the reconstructed analyte traces to identify at least a given one of the different subsets of the analyte samples as being associated with a PISA if a residual between the measured analyte trace and a reconstructed analyte trace generated by excluding the given one of the different subsets of the analyte samples exceeds a threshold.

15. The system of claim 10, wherein the Bayesian denoising algorithm includes a tunable regularization parameter that is tunable to produce a smoother reconstructed analyte trace as the regularization parameter varies by one of increasing or decreasing and to produce a reconstructed analyte trace that more closely follows the data samples in the measured analyte trace as the regularization parameter varies by the other of increasing or decreasing.

16. The system of claim 15, wherein the processor is further configured to execute the instructions to: adjust the tunable regularization parameter to an optimal value that increases detection of true positive PISAs and decreases detection of false positive PISAs.

17. The system of claim 16, wherein the tunable regularization parameter is adjusted for a particular measured analyte trace.

18. The system of claim 15, wherein the Bayesian denoising algorithm includes a tunable regularization parameter that is tunable to produce a smoother reconstructed analyte trace as the regularization parameter varies by one of increasing or decreasing and to produce a reconstructed analyte trace that more closely follows the data samples in the measured analyte trace as the regularization parameter varies by the other of increasing or decreasing.

19. The system of claim 18, wherein the processor is further configured to execute instructions to: retune the regularization parameter to decrease the regularization parameter by a scaling factor that is greater than zero and less than one to thereby increase sensitivity and decrease detection of false positive PISAs.

20. The system of claim 19, wherein the scaling factor is determined based on a population dataset.

21. The system of claim 10, wherein the processor is further configured to execute instructions to: adjust a confidence window parameter that determines a width of the confidence window to balance detection of true positive PISAs and avoidance of detection of false positive PISAs.
